(19) United States
(12) Patent Application Publication
(10) Pub. No.: US 2025/0381410 A1
(43) Pub. Date: Dec. 18, 2025

(54) NEUROSTIMULATION SYSTEM INCLUDING AN EXTERNAL PULSE GENERATOR

(71) Applicant: Axonics, Inc., Irvine, CA (US)

(72) Inventors: Faizal ABDEEN, Irvine, CA (US); Henry LEE, Irvine, CA (US); Xuechen HUANG, Irvine, CA (US); Guangqiang JIANG, Irvine, CA (US); Prabodh MATHUR, Irvine, CA (US); Pete Peterson, Irvine, CA (US); John MILLER, Irvine, CA (US); Dennis SCHROEDER, Irvine, CA (US)

(73) Assignee: Axonics, Inc., Irvine, CA (US)

(21) Appl. No.: 19/238,067

(22) Filed: Jun. 13, 2025

Related U.S. Application Data
(60) Provisional application No. 63/660,386, filed on Jun. 14, 2024.

(51) Int. Cl. A61N 1/372; A61N 1/378

(52) U.S. Cl. CPC A61N 1/37241; A61N 1/37229; A61N 1/3782; A61N 1/3787

(57) ABSTRACT

An external pulse generator (EPG) for stimulating a nerve includes a housing including an electrical contact configured to receive a lead type for stimulating the nerve. The housing includes a door covering the electrical contact. The door is configured to be nested within and latched to the housing. The door is configured to swing open to uncover the electrical contact. A receptacle is located on the housing and is configured to receive a first end of an electrical cable. The is configured to be connected to a second lead type for stimulating the nerve. The EPG includes a battery that carries a communication signal and is located between a circuit board and the housing. The EPG includes a ground pad connector extending through the housing. The ground pad connector is attached to a ground pad that is configured to be placed on a surface of a skin of the patient.

### BACKGROUND

Treatments that employ implanted neurostimulation systems have become more common in recent years. While such systems have shown promise in treating a number of chronic conditions, effectiveness of treatment may vary considerably between patients and viability of treatment may be difficult to determine before implantation. Although conventional methods of implantation often utilize preliminary testing with a temporary, partly implanted neurostimulation systems to assess viability of treatment, such systems may not provide an accurate representation of treatment with a fully implanted device. In addition, such systems are often bulky, uncomfortable and limit patient mobility, such that many patients elect not to receive a temporary system or a fully implanted system. In addition, many such temporary partly implanted systems may not operate in the same manner as their fully implanted counterparts due to differences between pulse generators or changes in position of the neurostimulation leads during conversion. Therefore, it is desirable to provide methods and devices for providing trial treatment systems that provide a more accurate representation of treatment, improve patient comfort and provide consistent treatment outcomes as compared to fully implanted neurostimulation systems.

### SUMMARY

This application relates to neurostimulation treatment systems, and in particular a neurostimulation treatment system having an EPG with a multi-purpose connector receptacle and affixation devices on which the EPG is releasably mounted and that are secured to the patient during a trial neurostimulation treatment. Typically, such a trial neurostimulation treatment includes a partly implanted neurostimulation lead extending to an external pulse generator for conducting a trial neurostimulation treatment for assessing viability of a fully implanted system. The system includes a partly implanted neurostimulation lead that extends from one or more implanted neurostimulation electrodes to an external pulse generator (EPG) supported in an affixation device secured to the patient. The trial period may be as little as four to seven days may extend two to four weeks or more.

In one disclosed embodiment, an external pulse generator is provided that includes an outer housing having at least one connector pad and a receptacle. Either the at least one connector pad or receptacle may be adapted for removably coupling with a proximal portion of an implantable neurostimulation lead to electrically couple the external pulse generator with one or more neurostimulation electrodes of the neurostimulation lead implanted at a target tissue. The external pulse generator further includes a pulse generator electrically coupled with the at least one connector pad and the receptacle, where the pulse generator is adapted for generating neurostimulation pulses to one or more neurostimulation electrodes of the lead. The receptacle may be configured to support a tined lead via a Percutaneous Extension (PE) cable. The connector pad may be configured to support a Peripheral Nerve Evaluation (PNE) lead (bilateral or unilateral). The external pulse generator may further include a battery electrically coupled to the pulse generator.

In one disclosed embodiment, an external pulse generator is provided that includes an outer housing having at least one door. The door may be fastened onto the outer housing is configured to latch onto the outer housing. The door is configured to push a PNE lead onto conductors of the external pulse generator.

In one disclosed embodiment, an external pulse generator is provided that includes an antenna system, wherein the signal carrying structure of the antenna structure may include a battery. The battery is further electrically coupled to the pulse generator to provide neurostimulation pulses to one or more neurostimulation electrodes of the lead. The antenna system further includes a primary signal carrier or an antenna active element. The battery may be a coin cell battery.

In one disclosed embodiment, an external pulse generator is provided that includes a ground pad connector configured to be attached to a ground pad. The ground pad connector may be integrated to the PCB of the external pulse generator. The external pulse generator may be configured to be placed on top of the ground pad.

In one disclosed embodiment, an external pulse generator is provided that includes a method and system configured to estimate state of charge (SoC) of the battery.

In one disclosed embodiment, an external pulse generator is provided that includes a boost converter configured to increase the output voltage of the battery. The external pulse generator may be configured to utilize the boost converter when the compliance voltage is higher than the battery voltage.

In one disclosed embodiment, an external pulse generator is provided without any buttons (physical or touch). The absence of buttons creates a smooth, unbroken exterior. This minimizes areas where bacteria or contaminants can accumulate, making the device easier to sterilize and reducing the risk of infection. Furthermore, buttons and touch interfaces are mechanical or electrical weak points prone to wear and tear, especially in demanding environments. Eliminating these elements increases the EPG's overall robustness. A sealed, buttonless design provides superior protection against water ingress, moisture, dust, and other contaminants that can damage internal components. Eliminating buttons and their associated components (e.g. switches, membranes, and/or a capacitive layer) can free up valuable internal space, allowing for a smaller overall device footprint. The lack of buttons can provide safety against accidental button presses that would alter the intended stimulation parameters.

In one disclosed embodiment, a round external pulse generator is provided without any sharp corners. Rounded edges distribute pressure more evenly against the skin, minimizing discomfort during prolonged wear. Sharp corners can create concentrated stress points on the user, thus a round EPG without sharp corners is more comfortable for prolonged use.

### BRIEF DESCRIPTION OF THE DRAWINGS

FIG. 1 is a schematic illustration of a trial neurostimulation system.

FIG. 2A is a simplified view of a trial neurostimulation system according to one configuration.

FIG. 2B is a simplified view of a trial neurostimulation system according to another configuration.

FIG. 3 is an isometric close up view of the external pulse generator shown in FIGS. 2A and 2B.

FIG. 4 is an isometric view of the doors of the external pulse generator.

FIG. 5 is an exploded view of the external pulse generator shown in FIGS. 2A and 2B.

FIG. 6 is an isolated isometric view of components of the external pulse generator.

FIG. 7 is an isolated isometric view of the PCB of the external pulse generator.

### DETAILED DESCRIPTION

Neurostimulation has been used for many years to treat a variety of conditions, from chronic pain, to erectile dysfunction and various urinary dysfunctions. While neurostimulation has proven effective in many applications, effective therapy often relies on consistently delivering therapeutic activation by one or more neurostimulation electrodes to particular nerves or targeted regions with a pulse generator. In recent years, fully implantable neurostimulation have become increasingly more commonplace. Although such implantable systems provide patients with greater freedom and mobility, the neurostimulation electrodes of such systems are sometimes challenging to adjust once they are implanted. The neurostimulation electrodes are typically provided on a distal end of an implantable lead that is advanced through a tunnel formed in a patient tissue.

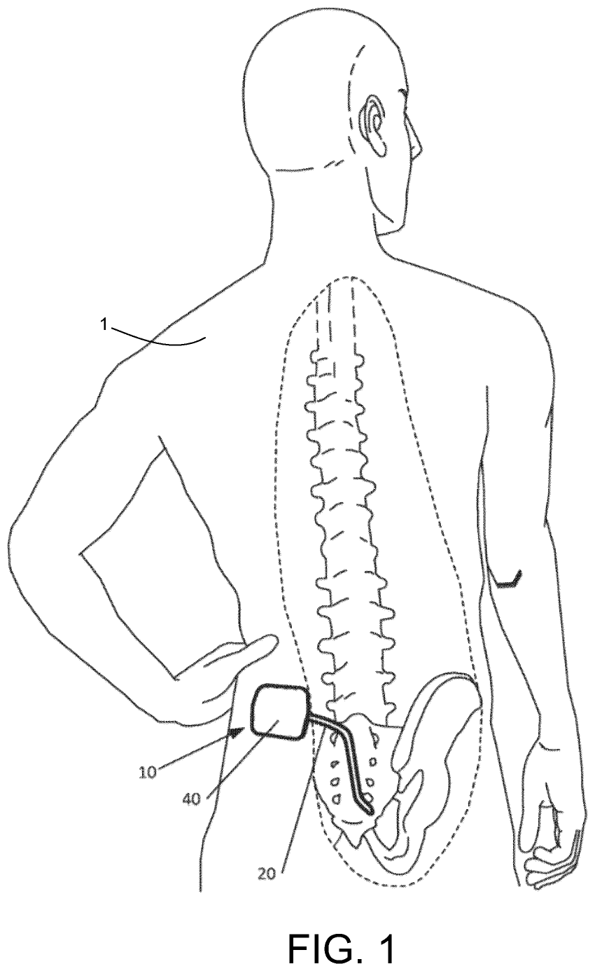

FIG. 1 schematically illustrates a trial neurostimulation system utilizing an EPG. Such a trial neurostimulation system may be used to assess viability of a fully implantable neurostimulation system. Implantable neurostimulation systems may be used in treating patients with, for example, chronic, severe, refractory neuropathic pain originating from peripheral nerves or various urinary and bowel dysfunctions. Implantable neurostimulation systems may be used to either stimulate a target peripheral nerve or the posterior epidural space of the spine. An implantable neurostimulation system includes an implanted pulse generator, typically implanted in a lower back region. The pulse generator may generate one or more non-ablative electrical pulses that are delivered to a nerve to control pain or cause some other desired effect.

The electrical pulses generated by the pulse generator are delivered to one or more nerves and/or to a target location via one or more leads that include one or more neurostimulation electrodes at or near the distal end. The leads may have a variety of shapes, may be a variety of sizes, and may be made from a variety of materials, which size, shape, and materials may be dictated by the application or other factors. In some applications, the leads may be implanted to extend along the spine or through one of the foramen of the sacrum, such as shown in FIG. 1, such as in sacral nerve stimulation. In other applications, the leads may be implanted in a peripheral portion of the patient's body, such as in the arms or legs, and may be configured to deliver one or more electrical pulses to the peripheral nerve such as may be used to relieve chronic pain.

In a conventional approach, prior to implantation of a permanent device, patients undergo an initial testing phase to estimate potential response to treatment. The first type of testing developed was percutaneous nerve evaluation (PNE). This testing procedure is done under local anesthesia, using a test needle to identify the appropriate sacral nerve(s). Once identified, a temporary wire lead is inserted through the test needle and left in place for 4 to 7 days. This temporary lead is connected to an external stimulator, which may be carried by patients in their pocket, secured against the skin under surgical dressings, or worn in a belt. The results of this test phase are used to determine whether patients are appropriate candidates for the permanent implanted device. For example, for overactive bladder, if patients show a 50 percent or greater reduction in symptom frequency, they are deemed eligible for the permanent device.

The second type of testing is a 2-stage surgical procedure. In Stage 1, a quadripolar-tined lead is implanted (stage 1). The tined lead may be connected to the EPG via a Percutaneous Extension (PE) cable. The testing phase may last as long as several weeks, and if patients show a specified reduction in symptom frequency, they may proceed to Stage 2 of the surgery, which is permanent implantation of the permanent neuromodulation device. The 2-stage surgical procedure has been used in various ways. These include its use instead of PNE, for patients who failed PNE, for patients with an inconclusive PNE, or for patients who had a successful PNE to further refine patient selection. Typically, in this 2-stage procedure, a percutaneous extension is utilized to connected the tined lead to the EPG in Stage 1.

Among the drawbacks associated with these conventional approaches, is the discomfort associated with wearing an EPG. The effectiveness of a trial period such as in PNE and Stage 1 trial periods are not always indicative of effective treatment with a permanent implanted system. Since effectiveness of treatment in a trial period may rely, in part, on a patient's subjective experience, the discomfort and inconvenience of wearing an EPG by the patient should be minimized so that the patient may resume ordinary daily activities without constant awareness of the presence of the EPG and treatment system. The comfort of the patient may be of particular importance in treatment of overactive bladder and erectile dysfunction, where a patient's awareness of the device could interfere with the patient's experience of symptoms associated with these conditions.

The system disclosed herein allows for improved assessment of efficacy during trial periods by providing a trial system having improved patient comfort so that patients may more easily recognize the benefits and effectiveness of treatment. The EPG disclosed herein is configured to deliver the therapy in substantially the same manner as the IPG in the permanent system such that the effects in permanent treatment should be more consistent with those seen in the trial system.

One or more properties of the electrical pulses may be controlled via a controller of the pulse generator. These properties may include, for example, the frequency, strength, pattern, duration, or other aspects of the timing and magnitude of the electrical pulses. These properties can include, for example, a voltage, a current, or the like. This control of the electrical pulses may include the creation of one or more electrical pulse programs, plans, or patterns, and in some embodiments, this control may include the selection of one or more pre-existing electrical pulse programs, plans, or patterns. In the embodiment depicted in FIG. 1, the neurostimulation system 10 includes a controller in the pulse generator having one or more pulse programs, plans, or patterns and/or to select one or more of the created pulse programs, plans, or patterns. The controller or processor may function with the assistance of an installed memory and may be adapted to provide instructions to and receive information from the other components of the neurostimulation system. The processor may include a microprocessor, such as a microprocessor from Intel® or Advanced Micro Devices®, or the like. The pulse generator may implement an energy storage feature, such as one or more capacitors or a battery.

Referring to FIG. 1, an example trial neurostimulation system 10 having an EPG 40 is illustrated. As shown, the neurostimulation system is adapted to stimulate a sacral nerve of the patient 1. The neurostimulation system 10 includes an EPG 40 attached to the lower back region, from which one or more neurostimulation leads 20 extends through a foramen of the sacrum to a position in which electrodes are disposed near the sacral root. The neurostimulation lead 20 may further include an anchor (not shown) disposed on a dorsal side of the sacrum. The anchor may be disposed on a ventral side of the sacrum, or within the foramen itself. The EPG 10 may be disposable and discarded after the trial is complete. Typically, the trial may last anywhere from 4 days to 8 weeks. Typically, an initial assessment may be obtained after 4-7 days and, if needed, effectiveness of treatment may be examined after a few weeks, typically about 2 weeks. EPG 10 may be held onto the skin by an adhesive patch. This patch may be an adhesive ground pad. The EPG 10 may be supported during the trial in various other manners, such as surgical tape, a belt, or holster.

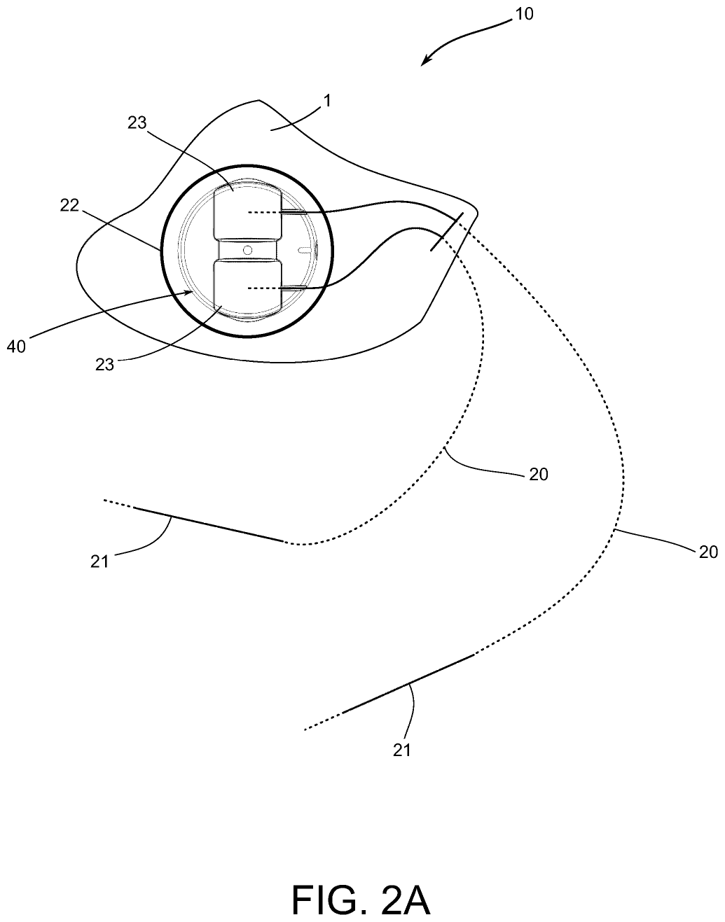

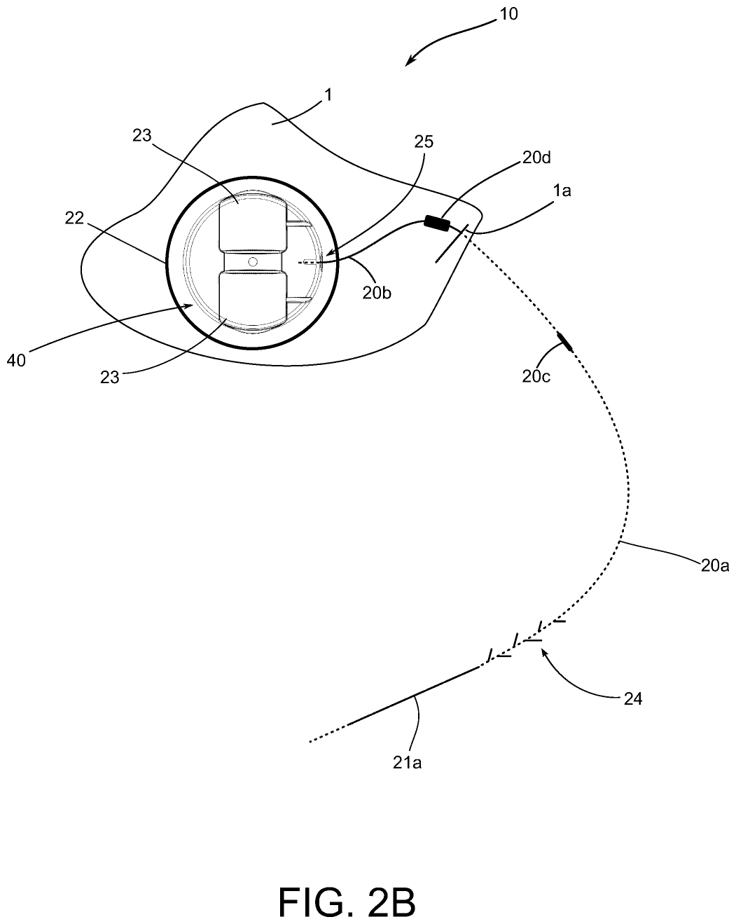

FIG. 2A shows an embodiment of neurostimulation system 10, similar to that in FIG. 1, in more detail. The neurostimulation leads 20 may include one or more a neurostimulation electrode(s) 21 at a distal end configured for PNE use and is electrically connected to the EPG 40.

The ground pad 22 may be connected to the EPG 40 via a fastening system. The ground pad 22 may be waterproof. The fastening system may be a snap on fastener. The ground pad is configured to attach onto the patient's body during the trial period. The ground pad 22 may be attached via an adhesive to the patient's body. The ground pad is typically placed on the skin near the area being treated and is used to complete the electrical circuit between the stimulator and the body. The purpose of the ground pad is to provide a low-resistance pathway for the electrical current to flow back to the stimulator, which helps to ensure that the current is delivered to the intended area of the body. Typically, the ground pad is separate from the stimulator and thus enveloping a larger surface area on the patient, which may cause annoyance and a greater amount of discomfort for the patient. The ground pad utilized herein, provides a smaller footprint and a more comfortable experience for the user.

In reference to FIG. 2A, the EPG 40 may be attached with PNE leads 20. Each PNE lead 20 is configured to be attached to contacts of the EPG 40 and latched within doors 23 of the EPG. Each PNE lead 20 includes a proximal end that is directly attached to one of the contacts (not shown) of the EPG 40 and a distal end which is the portion of the lead 20 located farthest from the EPG and including one or more electrodes 21. The proximal end of the lead is configured to be secured onto the contacts by the doors 23 of the EPG 40. The doors 23 latch onto the outer housing of the EPG 40.

FIG. 2B shows an alternative configuration of neurostimulation system 10, shown in FIG. 2A. In this configuration, the EPG 40 is equipped with a tined lead 20a comprising one or more tines 24. The lead 20a may include one or more a neurostimulation electrode(s) 21a at a distal end and is electrically connected to a cable 20b (e.g., a percutaneous extension cable) at the proximal end of lead 20a. The proximal end of the cable 20b is connected to the EPG 40 via receptacle 25. The cable 20b may include a connector 20c which is connected to the proximal end of the lead 20a. The tined lead 20a may be connected to the receptacle via a Percutaneous Extension (PE) cable where the cable is configured to be inserted to the receptacle 25. Lead 20a may be fully implanted inside the patient. Cable 20b extends from inside the patient and exiting out of the patient from an incision site la. The cable 20b may also include a regression stopper 20d, placed outside of the patient, where the regression stopper is configured to prevent regression of the proximal connector into a patient's body through the incision 1a.

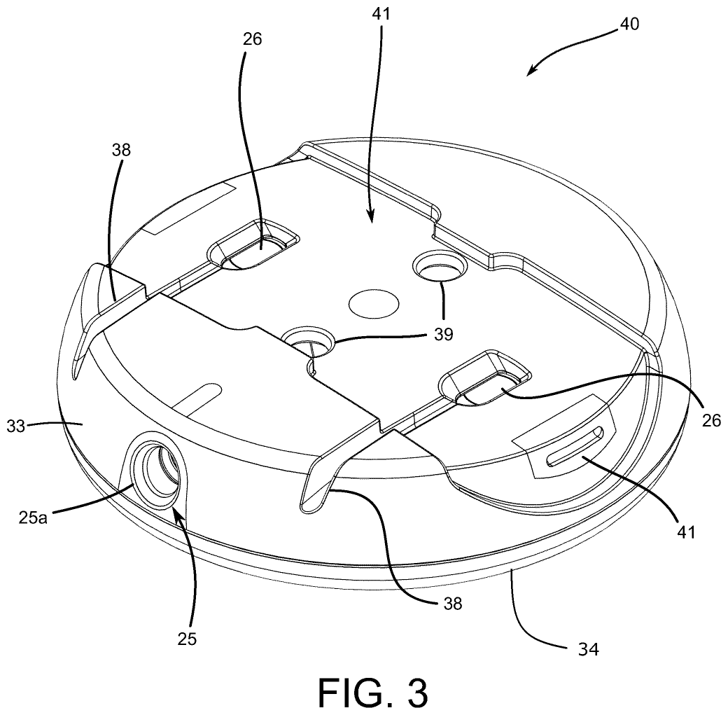

FIG. 3 shows a detailed view of the EPG 40 with doors 23 removed. The housing of the EPG includes a first portion 33 and a second portion 34. The EPG 40 may include two doors 23 covering contacts for the PNE leads as shown in FIG. 2A. The doors 23 are configured to cover conductor contacts for the PNE leads. The EPG 40 may be configured to connect to PNE leads 20 utilizing the contacts 26 or to the cable 20b having the connector 20c that connects to a tined lead 20a utilizing the receptacle 25. The receptacle 25 includes a receptacle opening 25a located on the first portion of the housing 33. The first portion of the housing 33 may also include guide slots 38 that are placed to aid in the alignment of the leads 20 to the contacts 26. The door may cover at least a part of the guide slots 38. The first portion of the housing 33 may also include attachment points 39 for doors 23 and a door recess 41 in which the latches (not shown) of the door 23 may attach to.

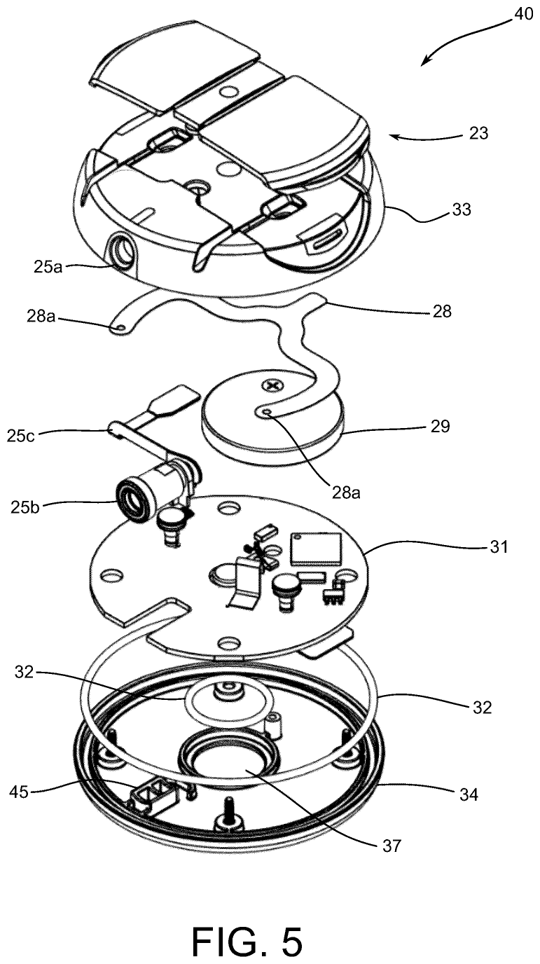

FIG. 4 shows the underside of the doors 23. The doors 23 may include attachment features 27, where the doors 23 are configured to be affixed (e.g. via friction fit or snap fit) onto the EPG 40. The doors may also contain projections 42 which help exert downward pressure on the proximal ends of the PNE leads, ensuring electrical connection with the contacts 26. The doors 23 are secured onto the EPG 40 with the latches 46 and contain hinges 47 near the attachment points to allow the door to open upwards. The doors 23 may also be transparent to ensure connection with PNE lead contacts 26.

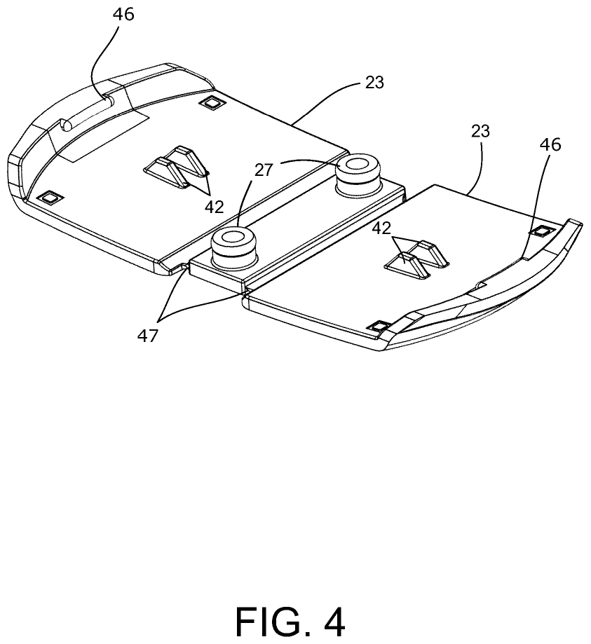

FIG. 5 shows a fully disassembled view of the EPG 40. The EPG 40 may further include includes doors 23, an antenna active element 28, a battery 29, receptacle grommet 25b, a main PCB 31, gaskets 32 which are all located between the first portion of the housing 33 and the second portion of the housing 34. The receptacle opening 25a is located on the first portion of the housing 33. The doors 23 may be configured to be nested within a cutout or depression of the first portion of the housing 33 such that the doors are flush against the outer surface of the first portion of the housing 33 when the doors are closed. The doors 23 may be made of a single monolithic piece of material. The first portion of the housing 33 and second portion of the housing 34 may be attached via screw or any other known fastening or attachment mechanisms. The receptacle 25 may include at least one flex cable 25c configured to establish an electrical connection between the electrical contacts of the receptacle and the PCB 31. The gaskets 32 ensure that the EPG is protected against unwanted liquid ingress, thus making the EPG 40 waterproof. One gasket may be placed on an outer perimeter of the second portion of the housing 34 and the other gasket may be placed on an inner perimeter of the second portion of the housing 34 adjacent to an opening 37 and provides a seal between the PCB 31 and the housing 34 at the opening 37. The receptacle 25 may also provide sealing for the housing via the grommet 25b. The receptacle grommet 25b may sit within a receptacle brace 45, located on the second portion of the housing 34, which holds the receptacle 25 in place.

The battery 29 may be part of the antenna system including the antenna active element 28. According to one exemplary embodiment, both the active element 28 and the battery 29 may be configured to carry a communication signal. The active antenna element 28 may be the primary signal carrying portion of the antenna system, but the battery 29 may be incorporated into the antenna system to increase the size (e.g., conductive area, volume and length) of the antenna system. The active element 28 is electrically connected to the battery 29 to create at least a portion of an antenna system configured for communication for the EPG 40.

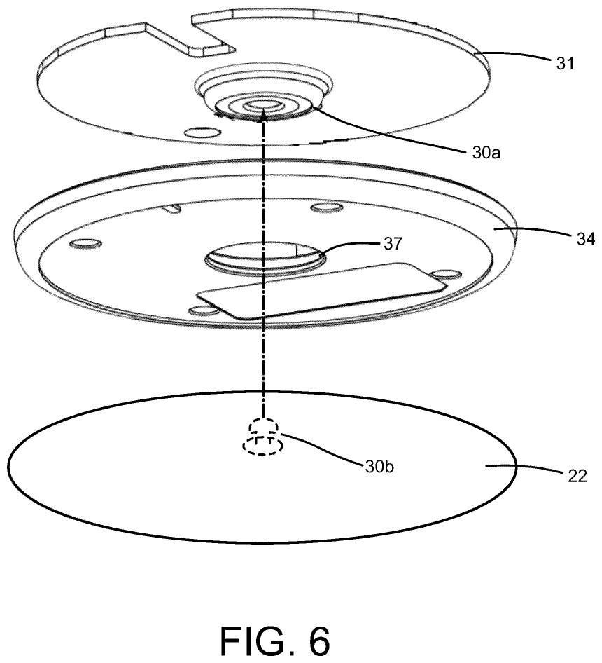

The antenna active element 28 is located between (preferably sandwiched) the battery 29 and the first portion of the housing 33 such that the antenna active element 28 is contact with both the battery 29 and the first portion of the housing 33. The antenna 28 may include attachment openings 28a which may be attached to the first portion of the housing 33 to secure the antenna 28. FIG. 6 shows the bottom of the PCB 31 and the top of pad 22. As mentioned above, the pad 22 may also include an adhesive surface for securing the EPG to the patient.

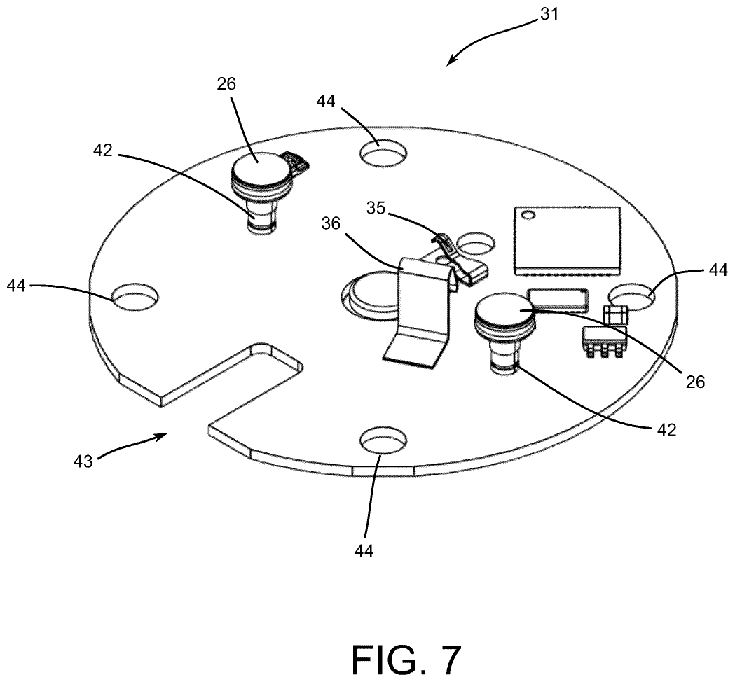

The PCB 31 may include an integrated female snap fitting 30a which is configured to attach to a male snap 30b fitting attached to the ground pad 22. The integrated female snap fitting 30a is configured to protrude through the second portion of the housing 34 through opening 37 as shown in FIG. 5. The integrated female snap fitting 30a may also be flush to the surface of housing 34. FIG. 7 shows the top of the PCB 31, the battery 29 is electrically connected to the PCB 31 and is suspended above the PCB 31 via a battery mount 35 which allows the battery 29 to be spaced apart from the circuit board and the female snap fitting 30a. The battery mount 35 maybe also be connected to the negative portion of the battery. The PCB 31 may also include a battery clip 36 that is contacting to the positive portion of the battery 29. The PCB 31 may also contain standoffs 42 which are connected to contacts 26. The PCB 31 may include a cutout portion 43 to accommodate the receptacle 25. Fastener holes 44 are incorporated into the PCB (31), facilitating the passage of fasteners from the second portion of the housing 34 to the first portion of the housing 33 to secure their connection.

The battery state of charge (SoC) may be estimated using known methods such as coulomb counting or other low voltage detection algorithms. Coulomb counting is a method of estimating the SoC of a battery by measuring the amount of electrical charge that flows in and out of the battery over time. The method involves integrating the current flowing into or out of the battery over time to determine the total amount of charge that has been transferred. This information may then be used to estimate the remaining capacity of the battery and to predict how long the battery will continue to provide power under a given load. The battery SoC estimation process will initiate when the EPG is assembled. During periods of inactivity after assembly (i.e. when stored on a shelf), the EPG will operate in a low power mode, and will periodically monitor for a control signal at a set low scan rate. Upon receiving the first control signal, the EPG transitions to a heightened power consumption state, characterized by a set high scan rate, to continuously monitor for subsequent control signals.

The external pulse generator 40 may be provided without any buttons (physical or touch). The external pulse generator may only be controlled by an external device such as a patient remote, a clinician programmer, or any other electronic devices capable of communicating with the telemetry system of the EPG 40.

The external pulse generator 40 may be provided without any sharp edges around the outer housing. It should be stated that the individual components of the EPG 40 may include corners, but when assembled together, the outer surface of the EPG 40 creates a substantially continuous surface that do not contain any sharp corners. That is, the outer housing is formed from multiple pieces joined together in a manner that eliminates sharp edges. Thus, the device is configured to minimize patient discomfort during the use of the EPG.

The EPG 40 may contain a boost converter without an accompanying buck converter. The boost converter is configured to allow the EPG to provide the correct compliance voltage for the stimulation when the voltage of the battery is insufficient. The exclusion of a buck converter contributes to the compact size of the EPG.

In the foregoing specification, the subject matter is described with reference to specific embodiments thereof, but those skilled in the art will recognize that the subject matter is not limited thereto. As used herein “user device” may refer to a device of any of a patient, a clinician or a specialist associated with the device provider or manufacturer. Various features and aspects of the above-described subject matter may be used individually or jointly. Further, the subject matter may be utilized in any number of environments and applications beyond those described herein without departing from the broader spirit and scope of the specification. The specification and drawings are, accordingly, to be regarded as illustrative rather than restrictive. The terms “comprising,” “including,” and “having,” as used herein, are specifically intended to be read as open-ended terms of art.

What is claimed is:

1. An external pulse generator (EPG) for stimulating a nerve comprising:
an outer housing;
a first electrical contact located on a surface of the outer housing, wherein the first electrical contact is configured to receive a first lead type;
at least one door covering the first electrical contact, wherein the door is configured to be nested within and latch to the outer housing; and
a receptacle, wherein the receptacle is configured to receive a first end of an electrical cable wherein the cable is configured to be connected to a second lead type at a second end of the electrical cable.

2. The external pulse generator of claim 1, wherein the first lead type is peripheral nerve evaluation (PNE) lead.

3. The external pulse generator of claim 1, wherein the electrical cable is a percutaneous extension (PE) cable.

4. The external pulse generator of claim 1, wherein the device is absent of any buttons.

5. The external pulse generator of claim 1, wherein the device is assembled such that the components of the EPG create a substantially continuous surface configured to minimize patient discomfort during the use of the EPG.

6. The external pulse generator of claim 1, further comprising a pulse generator disposed within the outer housing and electrically coupled to the first electrical contact and the receptacle, the pulse generator configured to generate neurostimulation pulses.

7. The external pulse generator of claim 1, wherein the at least one door comprises at least one projection on an underside thereof, the at least one projection configured to exert pressure on the first lead type when the door is in a closed position to ensure electrical connection with the first electrical contact.

8. The external pulse generator of claim 1, wherein the outer housing further comprises guide slots adjacent to the first electrical contact to aid in alignment of the first lead type with the first electrical contact.

9. The external pulse generator of claim 6, further comprising a battery disposed within the outer housing and electrically coupled to the pulse generator.

10. The external pulse generator of claim 9, wherein the battery forms part of an antenna system of the EPG configured for wireless communication.

11. The external pulse generator of claim 10, wherein the antenna system further comprises an antenna active element electrically coupled to the battery, wherein the antenna active element is a primary signal carrying portion of the antenna system.

12. The external pulse generator of claim 1, further comprising a ground pad connector on an external surface of the outer housing, the ground pad connector configured for electrical and mechanical attachment to an external ground pad.

13. The external pulse generator of claim 9, further comprising circuitry configured to estimate a state of charge of the battery.

14. The external pulse generator of claim 13, wherein the circuitry is configured to estimate the state of charge using a coulomb counting method.

15. The external pulse generator of claim 9, further comprising a boost converter electrically coupled to the battery, the boost converter configured to increase an output voltage of the battery.

16. The external pulse generator of claim 15, wherein the EPG does not include a buck converter.

17. The external pulse generator of claim 1, wherein the outer housing has a generally round shape and is devoid of sharp external corners.

18. The external pulse generator of claim 1, further comprising at least one gasket disposed between mating portions of the outer housing to provide a seal against liquid ingress.

19. The external pulse generator of claim 1, wherein the receptacle includes a receptacle grommet to provide a seal around an opening in the outer housing associated with the receptacle.

20. The external pulse generator of claim 1, wherein the EPG is configured to be disposable after a trial neurostimulation period.

21. An external pulse generator (EPG) for stimulating a nerve comprising:
a housing including a first portion having a lead attachment mechanism configured to receive a lead for stimulating the nerve, and a second portion configured to be attached to the first portion;
a circuit board located within the housing;
an antenna system comprising:
a battery configured to power the external pulse generator located between the circuit board and first portion; and
wherein the battery is configured to carry a communication signal.

22. The external pulse generator of claim 21, wherein the antenna system further comprises an antenna active element electrically coupled to the battery, the antenna active element being a primary signal carrying portion of the antenna system.

23. The external pulse generator of claim 22, wherein the antenna active element is located between the battery and the first portion of the housing such that the antenna active element is in contact with both the battery and the first portion of the housing.

24. The external pulse generator of claim 21, wherein the lead attachment mechanism comprises at least one connector pad on a surface of the first portion of the housing.

25. The external pulse generator of claim 24, wherein the at least one connector pad is configured to receive a Peripheral Nerve Evaluation (PNE) lead.

26. The external pulse generator of claim 21, wherein the lead attachment mechanism comprises a receptacle in the first portion of the housing, the receptacle configured to receive a Percutaneous Extension (PE) cable.

27. An external pulse generator (EPG) for stimulating a nerve comprising:
a housing comprising a first housing portion and a second housing portion attached thereto, the first housing portion including an external surface for interfacing with a patient or lead;
a circuit board disposed within the housing;
a battery disposed within the housing, positioned between the circuit board and an inner surface of the first housing portion, the battery configured to power the external pulse generator; and
an antenna active element located between the battery and the inner surface of the first housing portion, the antenna active element being electrically connected to the battery to create at least a portion of an antenna system, and wherein the battery is also configured to carry a communication signal as part of the antenna system.

28. The external pulse generator of claim 27, wherein the first housing portion includes a lead attachment mechanism comprising at least one connector pad configured to receive a Peripheral Nerve Evaluation (PNE) lead.

29. The external pulse generator of claim 28, further comprising at least one door movably coupled to the first housing portion, the at least one door configured to cover the at least one connector pad.

30. The external pulse generator of claim 27, further comprising a ground pad connector integrated with the circuit board and protruding through an opening in the second housing portion.

31. The external pulse generator of claim 30, wherein the ground pad connector comprises a snap fitting.

32. The external pulse generator of claim 27, wherein the battery is a coin cell battery.

33. The external pulse generator of claim 27, further comprising a pulse generator on the circuit board, the pulse generator electrically coupled to the battery and to a lead attachment mechanism on the first housing portion.

34. An external pulse generator (EPG) for stimulating a nerve, comprising:
a two-part housing including a first housing portion and a second housing portion defining an internal cavity;
a main printed circuit board (PCB) disposed within the internal cavity;
a battery configured to power the external pulse generator, the battery being positioned within the internal cavity and suspended above the main PCB via a battery mount, the battery mount connected to a negative terminal of the battery;
a battery clip on the main PCB contacting a positive terminal of the battery; and
an antenna system configured for wireless communication, the antenna system including the battery as a signal carrying structure and an antenna active element electrically coupled to the battery, the antenna active element being a primary signal carrier for the antenna system.

35. The external pulse generator of claim 34, wherein the first housing portion includes a receptacle configured to receive a Percutaneous Extension (PE) cable, the PE cable configured to connect to a tined lead.

36. The external pulse generator of claim 34, further comprising a boost converter electrically coupled to the battery and disposed on the main PCB.

37. The external pulse generator of claim 34, wherein an exterior surface of the two-part housing is devoid of physical buttons and touch-sensitive input areas.

38. The external pulse generator of claim 34, further comprising at least one gasket disposed between the first housing portion and the second housing portion to provide a seal.

39. The external pulse generator of claim 34, further comprising circuitry on the main PCB configured to estimate a state of charge of the battery.

40. The external pulse generator of claim 34, wherein the external pulse generator is configured to be disposable.

41. An external pulse generator (EPG) for stimulating a nerve comprising:
an outer housing having two lead contacts configured to receive leads for stimulating the nerve;
a door attached to the outer housing, wherein the door is located within a recess of the outer housing; and
wherein the door is configured to cover at least one of the two lead contacts and is configured to swing open to uncover the at least one of the two lead contacts.

42. The external pulse generator of claim 41, wherein the door is configured to latch to the outer housing when in a closed position.

43. The external pulse generator of claim 41, wherein the door comprises at least one projection on an underside thereof, the at least one projection configured to exert pressure on a lead placed on the at least one of the two lead contacts when the door is in a closed position to maintain electrical connection.

44. The external pulse generator of claim 41, wherein the door is transparent.

45. The external pulse generator of claim 41, wherein the two lead contacts are configured to receive Peripheral Nerve Evaluation (PNE) leads.

46. The external pulse generator of claim 41, wherein the door comprises hinges facilitating the swinging open of the door.

47. An external pulse generator (EPG) for stimulating a nerve, comprising:
an outer housing defining a recess on an external surface thereof, the outer housing further comprising guide slots adjacent to the recess;
at least two distinct electrical lead contacts exposed on the outer housing within an area defined by the recess, the lead contacts configured for direct connection with proximal ends of neurostimulation leads aligned by the guide slots; and
a door movably coupled to the outer housing via hinges, the door being dimensioned to be nested within the recess and cover the guide slots when in a closed position, the door further comprising projections on an underside thereof to exert pressure on the neurostimulation leads when connected to the lead contacts, wherein the door is configured to cover the at least two distinct electrical lead contacts in the closed position and to latch to the outer housing.

48. The external pulse generator of claim 47, wherein the door is made of a single monolithic piece of material.

49. The external pulse generator of claim 47, further comprising a pulse generator disposed within the outer housing and electrically coupled to the at least two distinct electrical lead contacts.

50. The external pulse generator of claim 49, further comprising a battery disposed within the outer housing and electrically coupled to the pulse generator.

51. The external pulse generator of claim 50, wherein the battery forms part of an antenna system of the external pulse generator.

52. The external pulse generator of claim 47, wherein an exterior surface of the outer housing is devoid of physical buttons and touch-sensitive input areas.

53. The external pulse generator of claim 47, wherein the outer housing has a generally round shape and is devoid of sharp external corners.

54. An external pulse generator (EPG) for stimulating a nerve, comprising:
an outer housing;
a first lead interface comprising two connector pads on the outer housing configured to receive Peripheral Nerve Evaluation (PNE) leads;
a door attached to the outer housing, the door configured to cover the two connector pads, to swing open via hinges to provide access to the two connector pads, and to be located within a depression of the outer housing such that the door is substantially flush with an outer surface of the outer housing when closed; and
a second lead interface comprising a receptacle in the outer housing, distinct from the first lead interface, the receptacle configured to receive an extension cable for connection to a tined lead.

55. The external pulse generator of claim 54, further comprising a battery, wherein the battery is electrically coupled to an antenna active element, the antenna active element being a primary signal carrier of an antenna system.

56. The external pulse generator of claim 54, further comprising a ground pad connector on an external surface of the outer housing.

57. The external pulse generator of claim 54, further comprising circuitry configured to estimate a state of charge of a battery powering the external pulse generator.

58. The external pulse generator of claim 54, further comprising a boost converter.

59. The external pulse generator of claim 54, further comprising at least one gasket to seal the outer housing against liquid ingress.

60. The external pulse generator of claim 54, wherein the external pulse generator is disposable.

61. An external pulse generator (EPG) for stimulating a nerve of a patient comprising:
an outer housing;
a first electrical contact located on a surface of the outer housing, wherein the first electrical contact is configured to receive a first lead;
at least one door covering the first electrical contact, wherein the door is configured to be nested within and latch to the outer housing; and
a ground pad connector extending through the outer housing, wherein the ground pad connector is attached to a ground pad, and the ground pad is configured to be placed on a surface of a skin of the patient.

62. The external pulse generator of claim 61, further comprising a printed circuit board (PCB) disposed within the outer housing, wherein the ground pad connector is electrically integrated with the PCB.

63. The external pulse generator of claim 62, wherein the ground pad connector comprises a female snap fitting integrated with the PCB, and the ground pad comprises a male snap fitting configured to attach to the female snap fitting.

64. The external pulse generator of claim 61, wherein the ground pad includes an adhesive surface for securing the ground pad to the skin of the patient.

65. The external pulse generator of claim 61, wherein the ground pad is waterproof.

66. The external pulse generator of claim 61, wherein the first electrical contact is configured to receive a Peripheral Nerve Evaluation (PNE) lead.

67. An external pulse generator (EPG) for stimulating a nerve of a patient, comprising:
an outer housing having a first portion and a second portion, the second portion having an opening therethrough;
a printed circuit board (PCB) disposed within the outer housing;
a first electrical contact on an external surface of the first portion of the outer housing, configured to receive a first neurostimulation lead;
at least one door configured to cover the first electrical contact and latch to the first portion of the outer housing; and
a ground pad connector integrated with the PCB, the ground pad connector protruding through the opening in the second portion of the outer housing and configured for electrical and mechanical attachment to an external ground pad, wherein the external ground pad is adapted to be adhered to a patient's skin, and wherein a gasket provides a seal between the PCB and the housing at the opening.

68. The external pulse generator of claim 67, wherein the external pulse generator is configured to be placed on top of the ground pad when the ground pad is attached to the patient.

69. The external pulse generator of claim 67, further comprising a receptacle in the outer housing, distinct from the first electrical contact, the receptacle configured to receive a proximal end of an electrical cable for connection to a second neurostimulation lead.

70. The external pulse generator of claim 67, further comprising a battery disposed within the outer housing, wherein the battery forms part of an antenna system.

71. The external pulse generator of claim 70, wherein the antenna system further comprises an antenna active element electrically coupled to the battery.

72. The external pulse generator of claim 67, wherein an exterior surface of the outer housing is devoid of physical buttons and touch-sensitive input areas.

73. The external pulse generator of claim 67, wherein the outer housing has a generally round shape and is devoid of sharp external corners.

74. A neurostimulation system for trial stimulation of a patient, comprising:
an external ground pad having an adhesive surface for securing to a patient's skin and a first snap fitting component; and
an external pulse generator (EPG) comprising:
an outer housing configured to be releasably mounted on top of the external ground pad;
a printed circuit board (PCB) within the outer housing;
at least one electrical contact on the outer housing configured to receive a neurostimulation lead;
at least one door covering the at least one electrical contact, the door configured to be nested within and latch to the outer housing; and
a second snap fitting component integrated with the PCB and extending through the outer housing, the second snap fitting component configured to mechanically and electrically connect with the first snap fitting component of the external ground pad.

75. The system of claim 74, wherein the at least one electrical contact is configured to receive a Peripheral Nerve Evaluation (PNE) lead.

76. The system of claim 74, wherein the EPG further comprises a battery, and circuitry on the PCB configured to estimate a state of charge of the battery.

77. The system of claim 74, wherein the EPG further comprises a boost converter electrically coupled to a battery within the EPG.

78. The system of claim 74, wherein the EPG is disposable.

79. The system of claim 74, wherein the at least one door comprises at least one projection on an underside thereof, configured to exert pressure on the neurostimulation lead when the door is in a closed position.

80. The system of claim 74, wherein the at least one electrical contact comprises at least two electrical contacts located on the surface of the outer housing and covered by the at least one door.
